(19) United States
(12) Patent Application Publication
(10) Pub. No.: US 2026/0358319 A1
(43) Pub. Date: Nov. 12, 2026

(54) METHOD FOR ESTIMATING MANTLE-DERIVED VOLATILE CONTENT

(71) Applicant: China University of Petroleum (East China), Qingdao (CN)

(72) Inventors: Ziqi Feng, Qingdao (CN); Yangming Li, Qingdao (CN); Haohao Wang, Qingdao (CN); Hao Li, Qingdao (CN); Yaqi Luo, Qingdao (CN); Wei Wang, Qingdao (CN); Mingxiang Yang, Qingdao (CN); Geyu Pang, Qingdao (CN)

(21) Appl. No.: 19/441,699

(22) Filed: Jan. 6, 2026

(30) Foreign Application Priority Data
May 12, 2025 (CN) 202510602685.5

(51) Int. Cl. G01N 33/24; G01N 33/00; G06F 17/11

(52) U.S. Cl. CPC G01N 33/24; G01N 33/004; G06F 17/11

(57) ABSTRACT

A method for estimating mantle-derived volatile content includes the steps of: acquiring sampling data from a target area; dividing the target area into core, intermediate, and peripheral zones of a tectono-thermal response zone in descending order of non-hydrocarbon carbon dioxide (CO₂) content in associated natural gas; calculating a proportion of CO₂ sourced from mantle-derived volatiles for each zone; and determining the mantle-derived volatile content of the target area based on the proportion and the corresponding non-hydrocarbon CO₂ content in the associated natural gas of each zone. The present disclosure employs different methods to calculate the proportion of CO₂ sourced from mantle-derived volatiles for zones with varying CO₂ contents, effectively enhancing the accuracy of mantle-derived volatile content estimation. A migration range of mantle-derived volatiles can be defined with greater accuracy, defining exploration targets for strategic helium (He) and hydrogen (H₂) resources in oil and gas fields and reducing exploration risks.

### CROSS-REFERENCE TO RELATED APPLICATION

This application claims priority of Chinese Patent Application No. 202510602685.5, filed on May 12, 2025, the entire contents of which are incorporated herein by reference.

### TECHNICAL FIELD

The present disclosure belongs to the technical field of noble gas geochemistry research, and specifically relates to a method for estimating mantle-derived volatile content in a tectono-thermal response zone.

### BACKGROUND

Mantle-derived volatiles are volatile substances released from the Earth's interior mantle, primarily found in the epicenters of tectono-thermal events, with a composition dominated by carbon dioxide (CO₂) and nitrogen (N₂) and a close association with helium (He) and hydrogen (H₂). Among these volatiles, CO₂ is not only the core component of deep mantle-derived volatiles but also plays a key role in the migration of these volatiles.

In the deep mantle, CO₂ can coexist with He and H₂. Driven by tectonic activity, high-concentration mantle-derived CO₂ acts as a dominant carrier, transporting He and H₂ in the form of multiphase seepage or gas-carrying phases, and migrates upward through channels such as faults. During migration, as the mantle-derived CO₂ content decreases, a transport system transitions to a CO₂—N₂ mixed carrier phase. When a mantle-derived CO₂ signal drops below detectable levels, N₂ assumes the role as the sole transport agent, continuing to influence the migration of He and H₂. During this process, the dissolution and mineralization of CO₂ exert a significant influence on the enrichment of He and H₂. For example, in the petroliferous basins of eastern China, the formation of mantle-derived CO₂ gas reservoirs is closely associated with the enrichment of He. The dissolution and mineralization of CO₂ increase the relative concentration of He in the gas phase, thereby promoting its enrichment, which is beneficial for oil and gas field exploration and development.

Investigating the carbon isotopic behavior and evolutionary mechanisms during the migration of mantle-derived volatiles is of significant importance. Particularly in regions strongly influenced by mantle-derived fluids, such as those with deep-seated major faults or diapiric activity, such research not only contributes to a deeper understanding of the geochemical characteristics of natural gas but also assists in identifying the potential sources of He and H₂. It can provide key insights into the formation and enrichment of He and H₂ and offer a scientific basis for exploration, thereby helping mitigate exploration risks and promoting the development and utilization of these unconventional energy resources.

### SUMMARY

Based on the aforementioned background, an objective of the present disclosure is to provide a method for estimating mantle-derived volatile content. Specifically targeting oil and gas fields within geological settings characterized by tectono-thermal response zones, such as those with deep-seated faults, volcanic activity, or diapiric structures, the method can achieve precise characterization of mantle-derived volatile content, thereby providing more accurate data support for natural gas geochemical research and exploration.

To realize the aforementioned objective, the present disclosure employs the following technical solution: a method for estimating mantle-derived volatile content includes the steps of:
S100, acquiring sampling data from a target area, where the target area is a tectono-thermal response zone, the sampling data including helium-3 (³He) content, carbon isotope (δ¹³C) value, and non-hydrocarbon CO₂ content in associated natural gas;
S200, dividing the target area into a tectono-thermal response zone core, an intermediate tectono-thermal response zone, and a peripheral tectono-thermal response zone, in descending order of the non-hydrocarbon CO₂ content in associated natural gas;
S300, calculating a proportion of CO₂ sourced from mantle-derived volatiles for the tectono-thermal response zone core, the intermediate tectono-thermal response zone, and the peripheral tectono-thermal response zone; and
S400, determining the mantle-derived volatile content of the target area based on the proportion of CO₂ sourced from mantle-derived volatiles and the corresponding non-hydrocarbon CO₂ content in the associated natural gas of each zone.

Further, in the tectono-thermal response zone core, the non-hydrocarbon CO₂ content in the associated natural gas is greater than 50%; in the intermediate tectono-thermal response zone, the non-hydrocarbon CO₂ content in the associated natural gas ranges from 5% to 50%; and in the peripheral tectono-thermal response zone, the non-hydrocarbon CO₂ content in the associated natural gas is less than 5%.

Further, the proportion of CO₂ sourced from mantle-derived volatiles in the peripheral tectono-thermal response zone is calculated based on the following formulas:

(C/A)_{MEA}=α/f1_{VOL}+β/f1_{DEC}+γ/f1_{DIC}+ε/f1_{EVO}+ϵ/f1_{DOC} (1)

(B/C)_{MEA}=α/f2_{VOL}+β/f2_{DEC}+γ/f2_{DIC}+ε/f2_{EVO}+ϵ/f2_{DOC} (2)

$$σ = - ( \frac{x}{f {1}_{DEC}} + \frac{1 - x}{f {1}_{DIC}} ) / 40 \qquad ( 3 )$$

β=σx (4)

γ=σ(1−x) (5)

$$τ = ( \frac{y}{f {1}_{EVO}} + \frac{1 - y}{f {1}_{DOC}} ) = 2 \qquad ( 6 )$$

ε=τy (7)

ϵ=τ(1−y) (8)

σ+τ=1−α (9)
where A represents a normalized δ¹³C value, B represents the ³He content, and C represents the non-hydrocarbon CO₂ content in the associated natural gas; and a subscript MEA represents a measured sample value;
VOL, DEC, DIC, EVO, and DOC refer to mantle-derived volatiles, crustal-derived carbonate mineral thermal decomposition, dissolution inorganic carbon (DIC) from basement carbonate minerals, organic matter thermal evolution, and shallow fluid dissolved organic carbon (DOC), respectively;
α, β, γ, ε, and ϵ refer to the proportion of CO₂ sourced from mantle-derived volatiles, a proportion of CO₂ sourced from crustal-derived carbonate mineral thermal decomposition, a proportion of CO₂ sourced from DIC from basement carbonate minerals, a proportion of CO₂ sourced from organic matter thermal evolution, and a proportion of CO₂ sourced from shallow fluid DOC, respectively; σ and τ represent a proportion of inorganic-sourced CO₂ and a proportion of organic-sourced CO₂, respectively; and x and y are predefined normalized variables; and
values for f1_{VOL}, f1_{DEC}, f1_{DIC}, f1_{EVO}, and f1_{DOC} are −0.15‰, 0.5‰, −0.5‰, −0.029‰, and −0.025‰, respectively; and values for f2_{VOL}, f2_{DEC}, f2_{DIC}, f2_{EVO}, and f2_{DOC} are 1.5×10⁹, 0.9×10¹³, 1.5×10¹³, 0.8×10¹³, and 1.2×10¹³, respectively.

Further, the proportion of CO₂ sourced from mantle-derived volatiles in the intermediate tectono-thermal response zone is calculated based on the following formulas:

(C/A)_{MEA}=α/f3_{VOL}+β/f3_{DEC}+γ/f3_{DIC}+ε/f3_{EVO} (10)

(B/C)_{MEA}=α/f4_{VOL}+β/f4_{DEC}+γ/f4_{DIC}+ε/f4_{EVO} (11)

$$σ = - ( \frac{x}{f {3}_{DEC}} + \frac{1 - x}{f {3}_{DIC}} ) / 30 \qquad ( 12 )$$

β=σx (13)

γ=σ(1−x) (14)

σ+ε=1−α (15)
where A represents the normalized δ¹³C value, B represents the ³He content, and C represents the non-hydrocarbon CO₂ content in the associated natural gas; and the subscript MEA represents the measured sample value;
VOL, DEC, DIC, and EVO refer to the mantle-derived volatiles, crustal-derived carbonate mineral thermal decomposition, DIC from basement carbonate minerals, and organic matter thermal evolution, respectively; and
α, β, γ, and ε refer to the proportion of CO₂ sourced from mantle-derived volatiles, the proportion of CO₂ sourced from crustal-derived carbonate mineral thermal decomposition, the proportion of CO₂ sourced from DIC from basement carbonate minerals, and the proportion of CO₂ sourced from organic matter thermal evolution; σ is the proportion of inorganic-sourced CO₂; x is the predefined normalized variable; values for f3_{VOL}, f3_{DEC}, f3_{DIC}, and f3_{EVO} are −0.15‰, 0.33‰, −0.5‰, and −0.067‰, respectively; and values for f4_{VOL}, f4_{DEC}, f4_{DIC}, and f4_{EVO} are 1.5×10⁹, 0.9×10¹³, 1.5×10¹³, and 0.8×10¹³, respectively.

Further, the proportion of CO₂ sourced from mantle-derived volatiles in the tectono-thermal response zone core is calculated based on the following formulas:

(C/A)_{MEA}=α/f5_{VOL}+β/f5_{DEC}+γ/f5_{DIC} (16)

(B/C)_{MEA}=α/f6_{VOL}+β/f6_{DEC}+γ/f6_{DIC} (17)

β+γ=1−α (18)
where A represents the normalized δ¹³C value, B represents the ³He content, and C represents the non-hydrocarbon CO₂ content in the associated natural gas; and the subscript MEA represents the measured sample value;
VOL, DEC, and DIC refer to the mantle-derived volatiles, crustal-derived carbonate mineral thermal decomposition, and DIC from basement carbonate minerals, respectively;
α, β, and γ refer to the proportion of CO₂ sourced from mantle-derived volatiles, the proportion of CO₂ sourced from crustal-derived carbonate mineral thermal decomposition, and the proportion of CO₂ sourced from DIC from basement carbonate mineral, respectively; and
values for f5_{VOL}, f5_{DEC}, and f5_{DIC} are −0.15‰, 0.5‰, and −0.2‰, respectively; and values for f6_{VOL}, f6_{DEC}, and f6_{DIC} are 1.5×10⁹, 0.9×10¹³, and 1.5×10¹³, respectively.

Further, a value of ¿ ranges from 0% to 50%.

Further, the mantle-derived volatile content is calculated based on the following formula:

Ø=αC (19)
where α is the proportion of CO₂ sourced from mantle-derived volatiles, C is the non-hydrocarbon CO₂ content in the associated natural gas of the corresponding zone, and Ø is the mantle-derived volatile content.

The present disclosure has the following beneficial effects.

Due to proximity to the centers of tectono-thermal events such as deep-seated faults and deep crustal diapirs, CO₂ content is exceptionally high and migrates directly upward. The CO₂ diffuses outward from the tectonic core through a “radiation-like” process, during which its content gradually decreases. Consequently, the CO₂ content can serve as an indicator of the relative distance from the center of the tectono-thermal event to the shallow subsurface. For oil and gas fields with deep-seated faults or diapiric structures, the present disclosure creatively utilizes the aforementioned phenomenon by dividing the target exploration area into the tectono-thermal response zone core, the intermediate tectono-thermal response zone, and the peripheral tectono-thermal response zone. The proportion of CO₂ sourced from mantle-derived volatiles is calculated using distinct methods for these different zones, thereby effectively enhancing the accuracy of mantle-derived volatile content estimation. Therefore, the method of the present disclosure can achieve more accurate delineation of a migration range of mantle-derived volatiles, precisely defining exploration targets for strategic He and H₂ resources in oil and gas fields and reducing exploration risks.

The present disclosure further provides dividing areas with a non-hydrocarbon CO₂ content greater than 50% into the tectono-thermal response zone core, areas with a non-hydrocarbon CO₂ content ranging from 5% to 50% into the intermediate tectono-thermal response zone, and areas with a non-hydrocarbon CO₂ content less than 5% into the peripheral tectono-thermal response zone. The mantle-derived volatile content is calculated through condition-setting in stages, and computational efficiency is enhanced in conjunction with MATLAB programming.

### BRIEF DESCRIPTION OF THE DRAWINGS

FIG. 1 shows a statistical chart of mantle-derived volatile contents in typical wells of a gas field A.

FIG. 2 shows a distribution diagram showing planar locations of typical wells in the gas field A.

### DETAILED DESCRIPTION

To clearly illustrate the objectives, technical solutions, and advantages of the embodiments of the present application, the technical solutions of the present application are clearly and comprehensively described below in conjunction with the accompanying drawings in the embodiments of the present application. Obviously, the embodiments described herein constitute only a part of the embodiments of the technical solutions of the present application, not all possible embodiments. Based on the embodiments described in the present application, all other embodiments obtained by those of ordinary skill in the art without creative efforts fall within the protection scope of the technical solutions of the present application.

### EMBODIMENT

A method for estimating mantle-derived volatile content in geological settings characterized by tectono-thermal response zones, such as deep-seated faults and diapiric structures, includes the following steps:
In step 1, sampling data from a target area is acquired. The target area is defined as a tectono-thermal response zone. The sampling data includes relevant natural gas geochemical information, specifically including He content and its isotopic content (e.g., ³He content), δ¹³C value, and non-hydrocarbon CO₂ content in associated natural gas. In this embodiment, typical wells from a gas field A, which is characterized by tectono-thermal events, are selected for implementation, with the gas field A serving as the target area.
In step 2, based on the non-hydrocarbon CO₂ content in the associated natural gas, the target area is classified into three categories: a tectono-thermal response zone core, where the non-hydrocarbon CO₂ content is greater than 50%; an intermediate tectono-thermal response zone, where the non-hydrocarbon CO₂ content ranges from 5% to 50%; and a peripheral tectono-thermal response zone, where the non-hydrocarbon CO₂ content is less than 5%.
In step 3, when located in the peripheral tectono-thermal response zone with a non-hydrocarbon CO₂ content of less than 5%, a proportion of CO₂ sourced from mantle-derived volatiles is calculated based on the following formulas:

(C/A)_{MEA}=α/f1_{VOL}+β/f1_{DEC}+γ/f1_{DIC}+ε/f1_{EVO}+ϵ/f1_{DOC} (1)

(B/C)_{MEA}=α/f2_{VOL}+β/f2_{DEC}+γ/f2_{DIC}+ε/f2_{EVO}+ϵ/f2_{DOC} (2)

$$σ = - ( \frac{x}{f {1}_{DEC}} + \frac{1 - x}{f {1}_{DIC}} ) / 40 \qquad ( 3 )$$

β=σx (4)

γ=σ(1−x) (5)

$$τ = ( \frac{y}{f {1}_{EVO}} + \frac{1 - y}{f {1}_{DOC}} ) = 2 \qquad ( 6 )$$

ε=τy (7)

ϵ=τ(1−y) (8)

σ+τ=1−α (9)
where A represents a normalized δ¹³C value, B represents the ³He content, and C represents the non-hydrocarbon CO₂ content in the associated natural gas; and a subscript MEA represents a measured sample value; VOL, DEC, DIC, EVO, and DOC refer to mantle-derived volatiles, crustal-derived carbonate mineral thermal decomposition, DIC from basement carbonate minerals, organic matter thermal evolution, and shallow fluid DOC, respectively; α, β, γ, ε, and ϵ refer to the proportion of CO₂ sourced from mantle-derived volatiles, a proportion of CO₂ sourced from crustal-derived carbonate mineral thermal decomposition, a proportion of CO₂ sourced from DIC from basement carbonate minerals, a proportion of CO₂ sourced from organic matter thermal evolution, and a proportion of CO₂ sourced from shallow fluid DOC, respectively; σ and τ represent a proportion of inorganic-sourced CO₂ and a proportion of organic-sourced CO₂, respectively; x and y are predefined normalized variables; values for f1_{VOL}, f1_{DEC}, f1_{DIC}, f1_{EVO}, and f1_{DOC} are −0.15‰, 0.5‰, −0.5‰, −0.029‰, and −0.025‰, respectively; and values for f2_{VOL}, f2_{DEC}, f2_{DIC}, f2_{EVO}, and f2_{DOC} are 1.5×10⁹, 0.9×10¹³, 1.5×10¹³, 0.8×10¹³, and 1.2×10¹³, respectively. To ensure the accuracy and rationality of the final results, a value of ε is constrained to a range of 0% to 50%.

When located in the intermediate tectono-thermal response zone with a non-hydrocarbon CO₂ content of 5% to 50%, a proportion of CO₂ sourced from mantle-derived volatiles is calculated based on the following formulas:

(C/A)_{MEA}=α/f3_{VOL}+β/f3_{DEC}+γ/f3_{DIC}+ε/f3_{EVO} (10)

(B/C)_{MEA}=α/f4_{VOL}+β/f4_{DEC}+γ/f4_{DIC}+ε/f4_{EVO} (11)

$$σ = - ( \frac{x}{f {3}_{DEC}} + \frac{1 - x}{f {3}_{DIC}} ) / 30 \qquad ( 12 )$$

β=σx (13)

γ=σ(1−x) (14)

σ+ε=1−α (15)
where A represents the normalized δ¹³C value, B represents the ³He content, and C represents the non-hydrocarbon CO₂ content in the associated natural gas; and the subscript MEA represents the measured sample value. VOL, DEC, DIC, and EVO refer to the mantle-derived volatiles, crustal-derived carbonate mineral thermal decomposition, DIC from basement carbonate minerals, and organic matter thermal evolution, respectively. α, β, γ, and ε refer to the proportion of CO₂ sourced from mantle-derived volatiles, the proportion of CO₂ sourced from crustal-derived carbonate mineral thermal decomposition, the proportion of CO₂ sourced from DIC from basement carbonate minerals, and the proportion of CO₂ sourced from organic matter thermal evolution; σ is the proportion of inorganic-sourced CO₂; x is the predefined normalized variable; values for f3_{VOL}, f3_{DEC}, f3_{DIC}, and f3_{EVO} are −0.15‰, 0.33‰, −0.5‰, and −0.067‰, respectively; and values for f4_{VOL}, f4_{DEC}, f4_{DIC}, and f4_{EVO} are 1.5×10⁹, 0.9×10¹³, 1.5×10¹³, and 0.8×10¹³, respectively.

When located in the tectono-thermal response zone core with a non-hydrocarbon CO₂ content of less than 50%, a proportion of CO₂ sourced from mantle-derived volatiles is calculated based on the following formulas:

(C/A)_{MEA}=α/f5_{VOL}+β/f5_{DEC}+γ/f5_{DIC} (16)

(B/C)_{MEA}=α/f6_{VOL}+β/f6_{DEC}+γ/f6_{DIC} (17)

β+γ=1−α (18)
where A represents the normalized δ¹³C value, B represents the ³He content, and C represents the non-hydrocarbon CO₂ content in the associated natural gas; and the subscript MEA represents the measured sample value. VOL, DEC, and DIC refer to the mantle-derived volatiles, crustal-derived carbonate mineral thermal decomposition, and DIC from basement carbonate minerals, respectively. α, β, and γ refer to the proportion of CO₂ sourced from mantle-derived volatiles, the proportion of CO₂ sourced from crustal-derived carbonate mineral thermal decomposition, and the proportion of CO₂ sourced from DIC from basement carbonate mineral, respectively. Values for f5_{VOL}, f5_{DEC}, and f5_{DIC} are −0.15‰, 0.5‰, and −0.2‰, respectively; and values for f6_{VOL}, f6_{DEC}, and f6_{DIC} are 1.5×10⁹, 0.9×10¹³, and 1.5×10¹³, respectively.

In step 4, based on the MATLAB software, the aforementioned calculation formulas are coded and linked to a database. The proportion of CO₂ sourced from mantle-derived volatiles is calculated for the tectono-thermal response zone core, the intermediate tectono-thermal response zone, and the peripheral tectono-thermal response zone, as detailed below:
the Database Toolbox in MATLAB is used for connecting to a database (such as MySQL or SQLite) that stores data. Structured query language (SQL) query statements are employed to extract, for different well names, the non-hydrocarbon CO₂ content, ³He content, and normalized δ¹³C value from the associated natural gas. Specific operations include:

```
  % Connect to the database
  conn = database(‘database_name’, ‘username’, ‘password’, ‘Vendor’, ‘Server’,
‘Port’);
  % Query data
  sqlquery = ‘SELECT well_name, CO2, He3, delta13C FROM carbon_data’;
  data = fetch(conn, sqlquery);
  % Close the connection
  close(conn);
```

based on the values of non-hydrocarbon CO₂ content in the associated natural gas, the data are divided into three cases: CO₂>50%, CO₂ between 5% and 50%, and CO₂<5%. Data subsets for each case are extracted using logical indexing. Specific operations include:

```
% Extract the CO₂ column
CO₂ = data.CO₂;
% Create categorical logical indices
idx_high = CO2 > 50; % CO2 > 50%
idx_medium = CO2 >= 5 & CO2 <= 50; % CO2 between 5% and 50%
idx_low = CO2 < 5; % CO2 < 5%
% Extract the corresponding data subsets
try
  data = fetch(conn, sqlquery);
  A = data{:, 1}; % column 1 is A
  B = data{:, 2}; % column 2 is B
  C = data{:, 3}; % column 3 is C
catch
  error(‘Failed to read the file or the file format is invalid.’);
end
```

Constants and symbolic variables are defined to solve equation systems using iterative methods. Depending on the value of C, the corresponding equation system is selected for solution, obtaining the proportion of CO₂ sourced from mantle-derived volatiles for each zone. Specifically, the equation systems can be determined using the solve function, and the solution array for each variable can be output using the disp function. Specific operations include:
for i=1: num rows
   % Current row's C/A and B/C ratios
   c_over_a=C_over_A(i);
   b_over_c=B_over_C(i);
   c_value=C(i);
   % Adjust equations based on C value
   if c_value<=5
      % Original equation system
      coeff_matrix=[1/f(1), 1/f(2), 1/f(3), 1/f(4), 1/f(5);
         f(6), f(7), f(8), f(9), f(10);
         1, 1, 1, 1, 1];
      const_vector=[c_over_a; b_over_c; 1];
   elseif c_value>5 && c_value<50
      . . .
      % Check if the equation system is solvable
   if size(coeff_matrix, 1)<size (coeff_matrix, 2)
      % Solve using the least squares method
      solution=coeff_matrix\const_vector;
   else
      % Solve directly
      solution=coeff_matrix\const_vector;
End
. . .
   % Store results in the results matrix
   results(i,:)=[Q, W, E, R, T];

In step 5, the mantle-derived volatile content is calculated as follows:

Ø=αC (19)
where a is the proportion of CO₂ sourced from mantle-derived volatiles, Cis the non-hydrocarbon CO₂ content in the associated natural gas of the corresponding zone, and Ø is the mantle-derived volatile content. Since existing technologies cannot accurately calculate the mantle-derived volatile content, and CO₂ is the predominant component within mantle-derived volatiles, the CO₂ content sourced from mantle-derived volatiles can be used as an approximation for the total mantle-derived volatile content.

In step 6, based on the quantitative calculation results of mantle-derived volatile content, and in combination with the wells' planar locations and sampling depths within the tectono-thermal response zone of the oil and gas field, the spatial distribution of mantle-derived volatiles is analyzed, and sweet spots are identified.

The sampling data and calculation results of this embodiment are presented in Table 1.

TABLE 1
Statistical table of sample information, test data,
and MATLAB calculation results for gas field A

| Well | ³He | δ¹³C_{CO}_{₂} | CO₂ | α | β | γ | ε | ∈ | ∅ | Depth |
|---|---|---|---|---|---|---|---|---|---|---|
| number | content | (‰, VPDB) | (%) | (%) | (%) | (%) | (%) | (%) | (%) | (m) |
| A1 | 2.17E−10 | −16.2 | 0.65 | 50.18 | 0.31 | 0.76 | 39.08 | 9.66 | 0.33 | 1230 |
| A2 | 1.85E−10 | −19.9 | 0.79 | 35.12 | 4.20 | 8.81 | 39.50 | 12.37 | 0.28 | 1250 |
| A2 | 2.55E−10 | −15.5 | 0.81 | 47.31 | 5.22 | 10.56 | 35.22 | 1.70 | 0.38 | 1280 |
| A1 | 2.82E−10 | −19.9 | 1.2 | 35.27 | 11.70 | 19.50 | 33.45 | 0.09 | 0.42 | 1290 |
| A3 | 8.16E−10 | −3.33 | 7.01 | 17.47 | 53.69 | 22.50 | 6.34 | | 1.22 | 1110 |
| A4 | 7.91E−11 | −5.94 | 7.23 | 1.63 | 60.27 | 22.27 | 15.83 | | 0.12 | 3390 |
| A5 | 1.12E−09 | −6.96 | 16.82 | 9.95 | 58.15 | 22.38 | 9.52 | | 1.67 | 1450 |
| A6 | 4.17E−10 | −2.47 | 17.01 | 3.66 | 63.38 | 22.06 | 10.89 | | 0.62 | 350 |
| A7 | 1.47E−09 | −4.63 | 22 | 10.01 | 59.38 | 22.32 | 8.29 | | 2.20 | 1340 |
| A7 | 1.47E−09 | −3.65 | 22 | 10.01 | 59.65 | 22.31 | 8.03 | | 2.20 | 1340 |
| A8 | 4.17E−10 | −0.79 | 33.58 | 1.85 | 65.21 | 21.91 | 11.03 | | 0.62 | 4150 |
| A9 | 9.18E−11 | −2.15 | 34.77 | 0.38 | 65.81 | 21.86 | 11.95 | | 0.13 | 1470 |
| A7 | 1.49E−09 | −4.51 | 41 | 5.44 | 62.58 | 22.13 | 9.85 | | 2.23 | 1200 |
| A10 | 4.72E−10 | −6.37 | 42.03 | 1.67 | 64.48 | 21.98 | 11.87 | | 0.70 | 1590 |
| A11 | 2.53E−10 | −3.40 | 44.70 | 0.83 | 65.46 | 21.89 | 11.82 | | 0.37 | 1220 |
| A11 | 2.53E−10 | −3.41 | 45.21 | 0.82 | 65.47 | 21.89 | 11.82 | | 0.37 | 1190 |
| A4 | 1.76E−11 | −0.42 | 49.5 | 0.04 | 66.37 | 21.80 | 11.80 | | 0.02 | 2790 |
| A11 | 5.95E−11 | −3.91 | 52.15 | 0.15 | 70.38 | 29.46 | | | 0.08 | 1060 |
| A2 | 2.09E−10 | −3.2 | 53.57 | 0.57 | 70.66 | 28.78 | | | 0.31 | 1350 |
| A2 | 5.37E−11 | −2.9 | 54.81 | 0.13 | 70.69 | 29.18 | | | 0.07 | 1380 |
| A3 | 1.09E−09 | −8.49 | 59.73 | 2.71 | 69.79 | 27.51 | | | 1.62 | 1250 |
| A3 | 2.04E−09 | −3.30 | 59.74 | 5.11 | 71.37 | 23.53 | | | 3.05 | 1250 |
| A12 | 1.32E−11 | −0.96 | 62.17 | 0.01 | 71.21 | 28.78 | | | 0.01 | 4140 |
| A13 | 2.19E−09 | −3.65 | 69.33 | 4.72 | 71.35 | 23.93 | | | 3.27 | 1730 |
| A14 | 2.73E−11 | −1.84 | 70.10 | 0.04 | 71.06 | 28.90 | | | 0.03 | 4040 |
| A14 | 2.63E−11 | −1.84 | 70.10 | 0.04 | 71.06 | 28.90 | | | 0.03 | 4040 |
| A14 | 5.41E−11 | −1.84 | 70.10 | 0.10 | 71.07 | 28.83 | | | 0.07 | 4040 |
| A15 | 1.32E−10 | −0.32 | 71.12 | 0.26 | 71.40 | 28.34 | | | 0.18 | 1590 |
| A15 | 2.12E−10 | −0.32 | 71.39 | 0.43 | 71.43 | 28.15 | | | 0.31 | 1600 |
| A16 | 7.08E−11 | −2.70 | 71.39 | 0.13 | 70.91 | 28.96 | | | 0.09 | 1470 |
| A15 | 2.32E−10 | −0.63 | 71.5 | 0.47 | 71.37 | 28.16 | | | 0.34 | 1620 |
| A16 | 2.32E−10 | −3.8 | 72.03 | 0.47 | 70.74 | 28.79 | | | 0.34 | 1500 |
| A7 | 1.49E−09 | −3.26 | 78 | 2.85 | 71.24 | 25.91 | | | 2.22 | 1200 |
| A6 | 4.17E−10 | −2.47 | 78.90 | 0.78 | 71.09 | 28.13 | | | 0.62 | 350 |
| A15 | 1.18E−10 | −6.90 | 80.66 | 0.20 | 70.24 | 29.56 | | | 0.16 | 2130 |
| A17 | 1.19E−10 | −6.90 | 80.66 | 0.20 | 70.24 | 29.56 | | | 0.16 | 2130 |
| A18 | 4.24E−09 | −4.05 | 85.40 | 7.43 | 71.81 | 20.76 | | | 6.35 | 4100 |
| A17 | 1.27E−10 | −6.00 | 85.82 | 0.20 | 70.46 | 29.34 | | | 0.17 | 1560 |
| A19 | 9.08E−11 | −2.70 | 85.90 | 0.14 | 71.00 | 28.86 | | | 0.12 | 1780 |
| A17 | 2.78E−10 | −6.60 | 86.67 | 0.46 | 70.41 | 29.13 | | | 0.40 | 1790 |
| A19 | 7.20E−11 | −2.89 | 86.95 | 0.11 | 70.97 | 28.92 | | | 0.10 | 1880 |
| A17 | 2.11E−10 | −6.20 | 87.98 | 0.34 | 70.47 | 29.19 | | | 0.30 | 1820 |
| A20 | 1.73E−10 | −6.6 | 88.29 | 0.28 | 70.40 | 29.32 | | | 0.25 | 1840 |
| A19 | 7.56E−11 | −3.32 | 88.37 | 0.11 | 70.91 | 28.98 | | | 0.10 | 2100 |
| A19 | 9.48E−11 | −2.83 | 88.93 | 0.14 | 70.99 | 28.86 | | | 0.12 | 2220 |
| A19 | 7.40E−11 | −2.83 | 88.93 | 0.11 | 70.99 | 28.90 | | | 0.10 | 2340 |

In Table 1, δ¹³C_{CO}_{₂} represents the normalized δ¹³C value, and CO₂ represents the non-hydrocarbon CO₂ content in the associated natural gas.

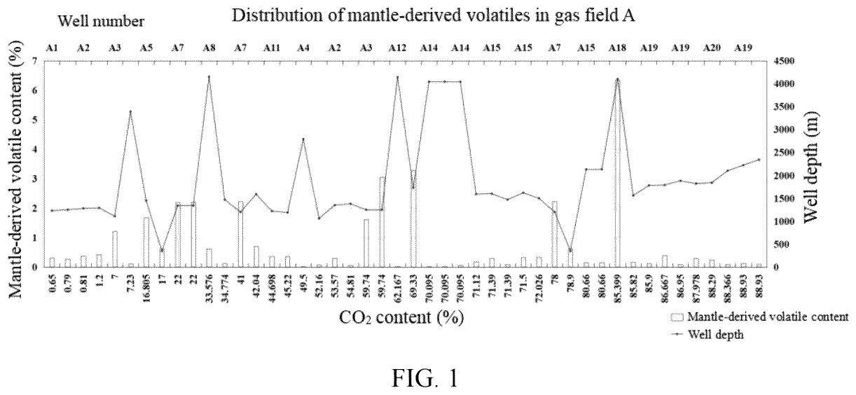

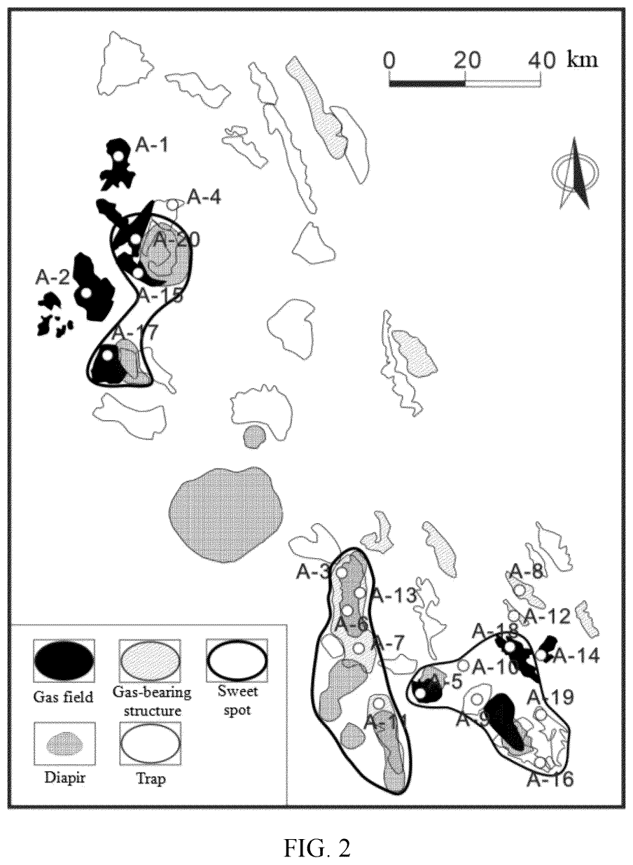

The calculated mantle-derived volatile content is shown in FIG. 1. The results indicate that, for the natural gas samples from typical wells in the gas field A, the mantle-derived volatile content ranges from 0.006% to 6.345%. A bar chart reveals multiple peaks. Verified by analysis of the wells' planar locations and sampling depths in the oil and gas field, the calculation results obtained by the method provided in the present disclosure can accurately capture the migration range and spatial distribution of mantle-derived volatiles within the oil and gas field (as shown in FIG. 2). Among them, the wells A3, A7, A13, and A18 exhibit higher mantle-derived volatile content compared to other wells, and can be prioritized as key exploration “sweet spots” for deep-seated He and H₂ resources, thereby reducing exploration risks and enhancing production efficiency.

1. A method for estimating mantle-derived volatile content, comprising the steps of:
S100, acquiring sampling data from a target area, where the target area is a tectono-thermal response zone, the sampling data comprising helium-3 (³He) content, carbon isotope (δ¹³C) value, and non-hydrocarbon carbon dioxide (CO₂) content in associated natural gas;
S200, dividing the target area into a tectono-thermal response zone core, an intermediate tectono-thermal response zone, and a peripheral tectono-thermal response zone, in descending order of the non-hydrocarbon CO₂ content in the associated natural gas;
S300, calculating a proportion of CO₂ sourced from mantle-derived volatiles for the tectono-thermal response zone core, the intermediate tectono-thermal response zone, and the peripheral tectono-thermal response zone; and
S400, determining the mantle-derived volatile content of the target area based on the proportion of CO₂ sourced from mantle-derived volatiles and the corresponding non-hydrocarbon CO₂ content in the associated natural gas of each zone, wherein
the proportion of CO₂ sourced from mantle-derived volatiles in the peripheral tectono-thermal response zone is calculated based on the following formulas:
(C/A)_{MEA}=α/f1_{VOL}+β/f1_{DEC}+γ/f1_{DIC}+ε/f1_{EVO}+ϵ/f1_{DOC} (1)
(B/C)_{MEA}=α/f2_{VOL}+β/f2_{DEC}+γ/f2_{DIC}+ε/f2_{EVO}+ϵ/f2_{DOC} (2)
$$σ = - ( \frac{x}{f {1}_{DEC}} + \frac{1 - x}{f {1}_{DIC}} ) / 40 \qquad ( 3 )$$
β=σx (4)
γ=σ(1−x) (5)
$$τ = ( \frac{y}{f {1}_{EVO}} + \frac{1 - y}{f {1}_{DOC}} ) = 2 \qquad ( 6 )$$
ε=τy (7)
ε=τ(1−y) (8)
σ+τ=1−α (9)
the proportion of CO₂ sourced from mantle-derived volatiles in the intermediate tectono-thermal response zone is calculated based on the following formulas:
(C/A)_{MEA}=α/f3_{VOL}+β/f3_{DEC}+γ/f3_{DIC}+ε/f3_{EVO} (10)
(B/C)_{MEA}=α/f4_{VOL}+β/f4_{DEC}+γ/f4_{DIC}+ε/f4_{EVO} (11)
$$σ = - ( \frac{x}{f {3}_{DEC}} + \frac{1 - x}{f {3}_{DIC}} ) / 30 \qquad ( 12 )$$
β=σx (13)
γ=ε(1−x) (14)
σ+ε=1−α (15)
the proportion of CO₂ sourced from mantle-derived volatiles in the intermediate tectono-thermal response zone is calculated based on the following formulas:
(C/A)_{MEA}=α/f5_{VOL}+β/f5_{DEC}+γ/f5_{DIC} (16)
(B/C)_{MEA}=α/f6_{VOL}+β/f6_{DEC}+γ/f6_{DIC} (17)
β+γ=1−α (18)
where A represents a normalized δ¹³C value, B represents the ³He content, and C represents the non-hydrocarbon CO₂ content in the associated natural gas; and a subscript MEA represents a measured sample value;
VOL, DEC, DIC, EVO, and DOC refer to mantle-derived volatiles, crustal-derived carbonate mineral thermal decomposition, dissolution inorganic carbon (DIC) from basement carbonate minerals, organic matter thermal evolution, and shallow fluid dissolved organic carbon (DOC), respectively;
α, β, γ, ε, and ϵ refer to the proportion of CO₂ sourced from mantle-derived volatiles, a proportion of CO₂ sourced from crustal-derived carbonate mineral thermal decomposition, a proportion of CO₂ sourced from DIC from basement carbonate minerals, a proportion of CO₂ sourced from organic matter thermal evolution, and a proportion of CO₂ sourced from shallow fluid DOC, respectively; σ and τ represent a proportion of inorganic-sourced CO₂ and a proportion of organic-sourced CO₂, respectively; and x and y are predefined normalized variables;
values for f1_{VOL}, f1_{DEC}, f1_{DIC}, f1_{EVO}, and f1_{DOC} are −0.15% ‰, 0.5‰, −0.5‰, −0.029‰, and −0.025‰, respectively; and values for f2_{VOL}, f2_{DEC}, f2_{DIC}, f2_{EVO}, and f2_{DOC} are 1.5×10⁹, 0.9×10¹³, 1.5×10¹³, 0.8×10¹³, and 1.2×10¹³, respectively.
values for f3_{VOL}, f3_{DEC}, f3_{DIC}, and f3_{EVO} are −0.15‰, 0.33‰, −0.5‰, and −0.067‰, respectively; and values for f4_{VOL}, f4_{DEC}, f4_{DIC}, and f4_{EVO} are 1.5×10⁹, 0.9×10¹³, 1.5×10¹³, and 0.8×10¹³, respectively; and
values for f5_{VOL}, f5_{DEC}, and f5_{DIC} are −0.15‰, 0.5‰, and −0.2‰, respectively; and values for f6_{VOL}, f6_{DEC}, and f6_{DIC} are 1.5×10⁹, 0.9×10¹³, and 1.5×10¹³ respectively.

2. The method for estimating mantle-derived volatile content according to claim 1, wherein in the tectono-thermal response zone core, the non-hydrocarbon CO₂ content in the associated natural gas is greater than 50%; in the intermediate tectono-thermal response zone, the non-hydrocarbon CO₂ content in the associated natural gas ranges from 5% to 50%; and in the peripheral tectono-thermal response zone, the non-hydrocarbon CO₂ content in the associated natural gas is less than 5%.

3. The method for estimating mantle-derived volatile content according to claim 1, wherein a value of ε ranges from 0% to 50%.

4. The method for estimating mantle-derived volatile content according to claim 1, wherein the mantle-derived volatile content is calculated based on the following formula:
Ø=αC (19)
where a is the proportion of CO₂ sourced from mantle-derived volatiles, C is the non-hydrocarbon CO₂ content in the associated natural gas of the corresponding zone, and Ø is the mantle-derived volatile content.
